(19) United States
(12) Patent Application Publication
(10) Pub. No.: US 2021/0113640 A1
(43) Pub. Date: Apr. 22, 2021

(54) COMPOSITION FOR PROMOTING HAIR GROWTH OR PREVENTING HAIR LOSS CONTAINING EXTRACT OF ASTER AGERATOIDES

(71) Applicant: KOREA INSTITUTE OF SCIENCE AND TECHNOLOGY, Seoul (KR)

(72) Inventors: Hyoung Ja KIM, Seoul (KR); Chang Bae JIN, Seoul (KR); Seon Hee SEO, Seoul (KR)

(21) Appl. No.: 17/035,391

(22) Filed: Sep. 28, 2020

(30) Foreign Application Priority Data
Oct. 16, 2019 (KR) 10-2019-0128516

(51) Int. Cl. A61K 36/28; A61P 17/14; A61Q 7/00; A61K 8/9789

(52) U.S. Cl. CPC A61K 36/28; A61P 17/14; A61K 2236/333; A61K 8/9789; A61Q 7/00

(57) ABSTRACT

Disclosed is a composition for promoting hair growth or preventing hair loss containing an Aster ageratoides alcohol extract or a solvent fraction obtained through fractionation therefrom as an active ingredient. The composition for promoting hair growth or preventing hair loss has excellent effects of inducing hair growth in a growth phase, preventing hair loss in a catagen phase and proliferating skin cells, and is thus capable of effectively overcoming a variety of problems caused by hair-growth promotion agents or hair-loss prevention agents.

### CROSS-REFERENCE TO RELATED APPLICATION

This application claims under 35 U.S.C. § 119(a) the benefit of priority to Korean Patent Application No. 10-2019-0128516 filed on Oct. 16, 2019, the entire contents of which are incorporated herein by reference.

### BACKGROUND

### (a) Technical Field

The present invention relates to a composition for promoting hair growth or preventing hair loss containing an Aster ageratoides extract or a fraction thereof as an active ingredient, and a cosmetic composition, health functional food composition or pharmaceutical composition containing an Aster ageratoides extract or a fraction thereof as an active ingredient.

### (b) Background Art

In addition to basic functions that are important in the human body, such as protecting the brain from external harmful materials and maintaining body temperature, hair also plays a cosmetically essential role as an indispensible part of the body that determines appearance. Hair is produced in hair follicles by continuous proliferation of scalp stromal cells, and has a hair cycle including various growth stages. Hair growth and loss are repeated according to a hair cycle including anagen (a growing phase) that accounts for 90% of normal hair, catagen (a regression phase) where growth stops and hair follicles atrophy, and telogen (a resting phase), in which hair bulbs are dried and become club hairs (Saitoh et al., 1970). In particular, during catagen (regression phase), apoptosis of a number of follicles progresses. As the telogen (resting phase) starts, the size of the hair follicles decreases (Buhl et al., 1990).

Alopecia (hair loss) has long been considered a series of phenomena of aging, but it has recently been revealed to be caused by various factors such as stress, westernized eating habits, and nutritional imbalance along with several genetic factors (Peters et al., 2006; Treb, 2002). The global population experiencing hair loss continues to increase, the age group experiencing hair loss is broadening to include younger persons in their 20s and 30s, and the proportion of women experiencing hair loss is also increasing rapidly.

A typical mechanism among the causes of hair loss is that testosterone is converted to dihydrotestosterone (DHT) by 5 alpha-reductase, which causes atrophy of hair follicles in the scalp, resulting in hair loss. With increasing age, DHT increases, protein synthesis of hair follicle cells is delayed, and as a result, the proportion of resting-phase hair follicles increases, and hair loss progresses rapidly (Adachi & Kano, 1970). Minoxidil agents for application and finasteride medications for oral administration are generally used as therapeutic agents for androgenic alopecia. Although the mechanism of action regarding the effect of minoxidil on hair growth is still unclear, it is known that an increase in nutrient supply through vasodilation and a potassium-channel-opening effect are involved in hair growth. Finasteride was developed as a therapeutic agent for prostatic hyperplasia, and is currently used as a therapeutic agent for hair loss. It is known that finasteride exhibits a hair-growth effect by lowering the concentration of DHT in the blood as an inhibitor of 5α-reductase (Dallob et al., 1994; Imperato-McGinley et al., 1974; Shapiro & Price, 1998).

However, minoxidil is reported to cause adverse reactions such as weight gain, edema, increased heart rate, angina, dermatitis, itching, erythema and skin dryness (Hageman et al., 2005; Mackay & Isles, 1981). Finasteride is reported to require continuous administration thereof so as to maintain the effect of promoting hair growth, and cause side effects such as sexual dysfunction in men and birth defects in pregnant women (Irwig, 2012; Rogers & Avram, 2008).

Accordingly, recently, in response to the demand for products that overcome these drawbacks and are safe and effective for continuous use, research has been actively conducted on natural products that are effective in preventing hair loss and promoting hair growth.

The above information disclosed in this Background section is only for enhancement of understanding of the background of the invention, and therefore it may contain information that does not form the prior art that is already known in this country to a person of ordinary skill in the art.

### PRIOR ART DOCUMENT

### Patent Document

(Patent Document 1) Korean Patent No. 10-1141236 entitled “Composition for oral administration containing an Aster ageratoides extract having whitening activity”

(Patent Document 2) Korean Patent No. 10-1083832 entitled “Composition for treating or preventing diabetic complications containing an Aster ageratoides extract”

### Non-Patent Documents

(Non-Patent Document 1) Saitoh et al., J. Invest. Dermatol. 54, 65-81, 1970

(Non-Patent Document 2) Buhl et al., Lab. Invest. 62, 104-107, 1990

(Non-Patent Document 3) Peters et al., Exp. Dermatol. 15, 1-13, 2006

(Non-Patent Document 4) TrOeb R. M. Experimental Gerontology 37, 981-990, 2002

(Non-Patent Document 5) Adachi & Kano. Biochem. Blophy. Res. Comm. 41, 884-890, 1970

(Non-Patent Document 6) Shapiro & Price. Dermatol. Clin. 16, 341-356, 1998

(Non-Patent Document 7) Dallob et al., J. Clin. Endocrinol. Metab. 79, 703-706, 1994

(Non-Patent Document 8) Imperato-McGinley et al., Science 186, 1213-1215, 1974

(Non-Patent Document 9) Hageman et al., Contact Dermatitis 53: 85-97, 2005

(Non-Patent Document 10) Mackay & Isles, Q. J. Med. 50, 175-190, 1981

(Non-Patent Document 11) Irwig M. S. J. Clin. Psychiatry 73, 1220-1223, 2012

(Non-Patent Document 12) Rogers & Avram. J. Am. Acad. Dermatol. 59, 567-568, 2008

### SUMMARY OF THE DISCLOSURE

In order to solve the above-described problems associated with the prior art, the present inventors searched for effects of promoting hair growth and preventing hair loss using natural raw materials, and found that an Aster ageratoides alcohol extract and solvent fractions obtained by fractionation therefrom exhibited effects of proliferating human dermal papilla cells (hDPCs), inducing hair anagen (growing phase) in animal models and preventing hair loss in artificially induced catagen (regression phase). Based on this finding, the present invention has been completed.

Thus, in an attempt to solve various problems of hair growth promoters or hair loss prevention agents reported in the prior art, it is an object of the present invention to provide a composition for promoting hair growth or preventing hair loss containing an Aster ageratoides alcohol extract or a fraction thereof as an active ingredient and a method of preparing the same.

The objects of the present invention are not limited to those described above. The objects of the present invention will be clearly understood from the following description, and can be implemented by the means defined in the claims and combinations thereof.

In order to accomplish the objects described above, the present invention provides the following composition.

In one aspect, the present invention provides a food composition, cosmetic composition or pharmaceutical composition for protecting the scalp and hair containing an Aster ageratoides extract or a solvent fraction thereof as an active ingredient by overcoming side effects of conventional hair-growth promotion agents and hair-loss prevention agents.

The present invention provides an Aster ageratoides solvent extract or an ethyl acetate fraction of the extract that exhibits an excellent effect of promoting hair growth by promoting the proliferation of human follicle dermal papilla cells.

In another aspect, the present invention provides a cosmetic composition and a pharmaceutical composition for promoting hair growth or preventing hair loss containing the Aster ageratoides alcohol extract or a solvent fraction obtained through fractionation therefrom as an active ingredient.

In one aspect, the present invention provides a composition for promoting hair growth or preventing hair loss containing an Aster ageratoides extract or a fraction thereof as an active ingredient.

In one aspect of the present invention, the Aster ageratoides extract is an extract of an aboveground or underground part of Aster ageratoides.

In one aspect of the present invention, the extract is an extract obtained through extraction using water, C₁-C₅ alcohol, acetone, an aqueous acetone solution or an aqueous C₁-C₅ alcohol solution.

In one aspect of the present invention, the concentrations of the aqueous C₁-C₅ alcohol solution and the aqueous acetone solution are each independently 10% to 90% (v/v).

In one aspect of the present invention, the fraction is an ethyl acetate fraction of an Aster ageratoides C₁-C₅ alcohol extract.

In one aspect of the present invention, the fraction is a butanol fraction of the Aster ageratoides C₁-C₅ alcohol extract.

In one aspect of the present invention, the extract is an extract of leaves of Aster ageratoides.

In one aspect of the present invention, the composition promotes hair growth or prevents hair loss by promoting the proliferation of human hair follicle dermal papilla cells.

In one aspect of the present invention, the composition promotes hair growth or prevents hair loss by inhibiting a regression phase (catagen) of hairs.

In one aspect of the present invention, the composition promotes hair growth or prevents hair loss by growing hair roots.

In one aspect of the present invention, the composition promotes hair growth or prevents hair loss by increasing a hair thickness or hair length.

In one aspect of the present invention, the composition promotes hair growth or prevents hair loss through free-radical scavenging or lipid peroxidation inhibition.

In one aspect of the present invention, the composition for promoting hair growth or preventing hair loss is a health functional food composition.

In one aspect of the present invention, the composition for promoting hair growth or preventing hair loss is a cosmetic composition.

In one aspect of the present invention, the composition for promoting hair growth or preventing hair loss is a pharmaceutical composition.

Other aspects and preferred embodiments of the invention are discussed infra.

### BRIEF DESCRIPTION OF THE DRAWINGS

The above and other features of the present invention will now be described in detail with reference to certain exemplary embodiments thereof, illustrated in the accompanying drawings which are given hereinbelow by way of illustration only, and thus are not limitative of the present invention, and wherein:

FIG. 1 is a graph showing cell proliferation effects when treating human hair follicle dermal papilla cells (hDPC) with minoxidil (10, 100 μM) as a positive control and with the composition according to the present invention containing an organic solvent fraction (5 or 50 μg/ml) obtained by solvent fractionation from an Aster ageratoides alcohol extract;

FIG. 2 is an image showing the effects on hair growth in C57BL/6 mice skin tissue after treatment with minoxidil (3%) as a positive control group and with the composition according to the present invention containing a 1% ethyl acetate fraction solution obtained by fractionation from the Aster ageratoides alcohol extract;

FIG. 3a is an image showing the effects on increases in thickness (A) and and FIG. 3b is an image showing the effects on increases in length (B) of hair grown from hair roots of C57BL/6 mice skin tissue after treatment with 3% minoxidil (b) as a positive control group and with the composition according to the present invention containing a 1% ethyl acetate fraction solution (c) obtained by fractionation from the Aster ageratoides alcohol extract;

FIG. 4 is a histological observation of vertical (A) and transversal (B) views showing the effects on hair follicle growth in C57BL/6 mice skin tissue after treatment with 3% minoxidil (b) as a positive control group and with the composition according to the present invention containing a 1% ethyl acetate fraction solution (c) obtained by fractionation from the Aster ageratoides alcohol extract;

FIG. 5 is an image showing an effect of prevention of hair loss in catagen-induced C57BL/6 mice skin tissue after inducing a catagen phase through treatment with 0.1% dexamethasone as a catagen inducer agent and then treating with 2% minoxidil as a positive control group and with the composition according to the present invention containing a 2% ethyl acetate fraction solution obtained by fractionation from the Aster ageratoides alcohol extract; and

FIG. 6 is an environmental scanning electron microscope (ESEM) image showing the density and thickness of hair grown from hair roots of catagen-induced C57BL/6 mice skin tissue after inducing a catagen phase through treatment with 0.1% dexamethasone as a catagen inducer agent and then treating with 2% minoxidil as a positive control group and with the composition according to the present invention containing a 2% ethyl acetate fraction solution obtained by fractionation from the Aster ageratoides alcohol extract.

### DETAILED DESCRIPTION

Unless the context clearly indicates otherwise, all numbers, figures and/or expressions that represent ingredients, reaction conditions, polymer compositions and amounts of mixtures used in the specification are approximations that reflect various uncertainties of measurement occurring inherently in obtaining these figures, among other things. For this reason, it should be understood that, in all cases, the term “about” should modify all numbers, figures and/or expressions. In addition, when numerical ranges are disclosed in the description, these ranges are continuous and include all numbers from the minimum to the maximum, including the maximum within the range, unless otherwise defined. Furthermore, when the range refers to an integer, it includes all integers from the minimum to the maximum including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include numbers such as 10%, 11%, 12% and 13% as well as 30%, and any sub-ranges of 10% to 15%, 12% to 18%, or 20% to 30%, as well as any numbers, such as 10.5%, 15.5% and 25.5%, between appropriate integers that fall within the range.

Hereinafter, the present invention will be described in detail.

In one aspect, the present invention provides a cosmetic composition and a pharmaceutical composition for protecting the scalp and hair containing an Aster ageratoides extract or a solvent fraction thereof as an active ingredient by overcoming side effects of conventional hair growth agents and hair loss prevention agents.

The present invention provides an Aster ageratoides solvent extract or an ethyl acetate fraction of the extract that exhibits an excellent effect of promoting hair growth by promoting the proliferation of human hair follicle dermal papilla cells.

In another aspect, the present invention provides a cosmetic composition and a pharmaceutical composition for promoting hair growth or preventing hair loss containing the Aster ageratoides alcohol extract or a solvent fraction fractionated therefrom as an active ingredient.

The present invention relates to an Aster ageratoides extract having excellent effects of proliferating human hair follicle dermal papilla cells, and promoting hair growth and increasing hair thickness in C57BL/6 mouse skin tissue.

According to one aspect of the present invention, the alcohol extract prepared from Aster ageratoides, and the solvent fraction and the Aster ageratoides ethyl acetate fraction obtained by fractionation therefrom are characterized by promoting the proliferation of human hair follicle dermal papilla cells.

In addition to this, the Aster ageratoides extract and fractions thereof according to the present invention have excellent free-radical scavenging activity and excellent inhibitory activity against lipid peroxide production, and thus can be useful for preventing or treating hair loss caused by oxidative stress.

In another aspect, the present invention provides a method for preparing the Aster ageratoides extract and solvent extracts according to the present invention including the following steps:

(Step 1) a first step of extracting Aster ageratoides with at least one extraction solvent selected from dichloromethane, acetone, an aqueous acetone solution, C₁-C₅ alcohol and an aqueous C₁-C₅ alcohol solution to obtain a solvent fraction; and

(Step 2) a second step of extracting the solvent extract obtained in step 1 with water and ethyl acetate to obtain an ethyl acetate fraction.

The Aster ageratoides used in the first step of obtaining the solvent extract may be any part of the plant growing aboveground or underground, and is preferably aboveground parts such as leaves, flowers, or stems of Aster ageratoides. The collected Aster ageratoides may be dried in the shade, or may be chopped, powderized or freeze-dried before use.

The extraction solvent used herein may be an ordinary organic solvent, and specifically may include at least one selected from dichloromethane, acetone, an aqueous acetone solution, C₁₋₅ alcohol and an aqueous C₁₋₅ alcohol solution. More specifically, the extraction solvent may be dichloromethane, acetone, methanol, butanol, a mixed solvent thereof, or an aqueous solution thereof containing 20 to 80% by volume of water.

The respective steps of the method of preparing the Aster ageratoides extract and the fractions thereof according to the present invention are described in detail below.

An extraction solvent is added in an amount of 0.1 to 5 L, preferably 0.5 to 1.0 L, per kg of Aster ageratoides, and is allowed to stand at room temperature for 4 to 5 days. The extraction may be performed 1 to 5 times, or may be performed a greater number of times as necessary. In addition, the temperature during extraction is preferably 10° C. to 100° C., and more preferably room temperature, but is not limited thereto. The extraction time is preferably 1 to 7 days, and more preferably 3 to 7 days, but is not limited thereto. The obtained extract is filtered, evaporated under reduced pressure, and dried to obtain a solvent extract. The evaporation under reduced pressure is preferably conducted using a vacuum rotary evaporator, but is not limited thereto. In addition, drying may be performed using one selected from reduced-pressure drying, vacuum drying, boiling drying, spray drying, room-temperature drying, and freeze drying, but is not limited thereto.

In the second step of obtaining a fraction, the solvent extract obtained above is extracted with water and ethyl acetate to obtain an ethyl acetate fraction.

More specifically, the ethyl acetate fraction may be obtained by adding 1 to 5 L, preferably 1.5 to 2.0 L, of water to 1 kg of the solvent extract, adding 0.1 to 5 L of ethyl acetate (EA), preferably 1.0 to 1.5 L, thereto, and sufficiently conducting extraction.

Further, in the present invention, the active fraction can be sufficiently obtained even when the ethyl acetate extract is obtained by directly extracting the Aster ageratoides with ethyl acetate without the first step of obtaining the solvent extract using the organic solvent. However, in order to obtain a higher-purity active fraction, it is preferable to sequentially perform the step of obtaining the ethyl acetate fraction after step 1) of obtaining the solvent extract.

In addition, the present invention is characterized by providing a cosmetic composition, pharmaceutical composition or health food composition for promoting hair growth and preventing and ameliorating hair loss containing an Aster ageratoides extract or a fraction thereof as an active ingredient.

That is, the effect of promoting hair growth was confirmed, and the effect of preventing hair loss in an artificially induced catagen (regression phase) was confirmed by conducting a biopsy on hair growth promotion due to the promotion of human hair follicle dermal papilla cell production, and hair thickening, hair growth and growth of hair roots in C57BL/6 mice by the Aster ageratoides extract and each of the solvent fractions thereof.

As can be seen from the following examples, when treating with the composition according to the present invention, effects of preventing hair loss and promoting hair growth were observed, and these effects are excellent to the extent of being competitive even with the minoxidil treatment group, used as a positive control group.

Hereinafter, various aspects of the present invention will be described.

In another aspect, the present invention provides a composition for promoting hair growth or preventing hair loss containing an Aster ageratoides extract or a fraction thereof as an active ingredient.

As used herein, the term “Aster ageratoides” is a perennial plant belonging to the Asteraceae family, and is found in Korea, China, Russia, and northern India. The name of the herb is Kalimeris yomena Kitam, and fresh leaves and sprouts of Aster ageratoides native to Korea are eaten raw, or are lightly boiled and eaten as vegetables. Flowers bloom in light purple in August to October, leaves are sharp, 10 to 14 cm long, 3 to 6 cm wide, and have rough surfaces and jagged edges, and fine hairs are present on stems and leaves. For this reason, it is also called “rough-surface Aster”. (Reference: Biodiversity on the Korean Peninsula, https://species.nibr.go.kr/home/mainHome.do?cont_link=009 &subMenu=009002&contCd=009002&ktsn=120000063730=Aster ageratoides). The prior art reports effects of Aster ageratoides; specifically, Patent Document 1 and Patent Document 2 report whitening activity and treatment and prevention of diabetes complications, respectively. However, it is not disclosed in the prior art that an extract of Aster ageratoides is effective in promoting hair growth and preventing hair loss.

As used herein, the term “extract” means any substance obtained by extracting ingredients from a natural product, regardless of the method of extraction or the type of ingredient. For example, broadly speaking, the extract includes a substance obtained by extracting an ingredient soluble in a solvent from a natural product using water or an organic solvent, a substance obtained by extracting only a specific ingredient from a natural product, or the like. In one embodiment of the present invention, the organic solvent is not particularly limited, and may be selected from C₁ to C₅ lower alcohols such as methanol, ethanol, isopropyl alcohol, n-propyl alcohol, n-butanol and isobutanol, polyhydric alcohols such as glycerol, ethylene glycol, propylene glycol and 1,3-butylene glycol, hydrocarbon solvents such as methyl acetate, ethyl acetate, benzene, n-hexane, diethyl ether, dichloromethane, chloroform, and non-polar organic solvents such as petroleum ether, methyl acetate, benzene, hexane, chloroform, methylene chloride, dimethyl ether, and ethyl acetate.

In one aspect of the present invention, the Aster ageratoides extract is an extract of an aboveground or underground part of Aster ageratoides.

In one aspect of the present invention, the extract is an extract obtained through extraction using water, C₁-C₅ alcohol, acetone, an aqueous acetone solution or an aqueous C₁-C₅ alcohol solution.

In one aspect of the present invention, the C₁-C₅ alcohol includes at least one selected from the group consisting of methanol, ethanol, isopropyl alcohol, n-propyl alcohol, n-butanol and isobutanol.

In one aspect of the present invention, the concentrations of the aqueous C₁-C₅ alcohol solution and the aqueous acetone solution are each independently 10% to 90% (v/v).

In one aspect of the present invention, the fraction is an ethyl acetate fraction of an Aster ageratoides C₁-C₅ alcohol extract.

In one aspect of the present invention, the fraction is a butanol fraction of an Aster ageratoides C₁-C₅ alcohol extract.

In one aspect of the present invention, the extract is an extract of leaves of Aster ageratoides.

In one aspect of the present invention, the Aster ageratoides extract or a fraction thereof may be present in an amount of 0.001 to 90% by weight based on the total weight of the composition. In one embodiment, the Aster ageratoides extract may be present in an amount of 0.001% by weight or more, 0.01% by weight or more, 0.1% by weight or more, 1% by weight or more, 1.1% by weight or more, 1.5% by weight or more, 2% by weight or more, 3% by weight or more, 5% by weight or more, 10% by weight or more, 20% by weight or more, or 30% by weight or more, based on the total weight of the composition. In addition, the Aster ageratoides extract may be present in an amount of 90% by weight or less, 85% by weight or less, 80% by weight or less, 70% by weight or less, 50% by weight or less, 40% by weight or less, 30% by weight or less, 20 by weight or less, 10% by weight or less, 5% by weight or less, 4% by weight or less, 3% by weight or less, 2% by weight or less, 1% by weight or less, 0.1% by weight or less or 0.05% by weight or less, based on the total weight of the composition.

In one aspect of the present invention, the composition promotes hair growth or prevents hair loss by promoting the proliferation of human hair follicle dermal papilla cells.

In one aspect of the present invention, the composition promotes hair growth or prevents hair loss by inhibiting a regression phase (catagen) of hairs.

In one aspect of the present invention, the composition promotes hair growth or prevents hair loss by growing hair roots.

In one aspect of the present invention, the composition promotes hair growth or prevents hair loss by increasing a hair thickness or hair length.

In one aspect of the present invention, the composition promotes hair growth or prevents hair loss by free-radical scavenging or lipid peroxidation inhibition.

In one aspect of the present invention, the composition for promoting hair growth or preventing hair loss is a health functional food composition.

The health food composition according to the present invention contains an extract of Aster ageratoides or a solvent fraction obtained through fractionation therefrom and there is no particular limitation as to the type thereof. Examples of the food include drinks, meat, sausages, breads, biscuits, rice cakes, Sunsik (Korean ready-to-eat food prepared from grains), chocolate, candy, snacks, confectioneries, pizza, ramen, other noodles, gums, dairy products including ice cream, various soups, beverages, alcoholic beverages, vitamin complexes, dairy products and processed dairy products, and include all other functional health foods in the conventional sense.

As an active ingredient, the extract of Aster ageratoides or the solvent fraction fractionated therefrom may be added alone to the food or may be used in conjunction with other foods or food ingredients, and may be suitably used according to conventional methods. The effective content may be appropriately determined according to the purpose of use (for prevention or amelioration), and may be present in a range of 0.001 to 70% by weight with respect to the total weight of the health food.

However, in the case of long-term intake for health and hygiene purposes or for health control, the amount may be below the above range, and the active ingredient may be used in an amount above the range, since there is no problem in terms of safety.

For example, in the case of preparing health beverages, the health drink may contain, in addition to the active ingredient, natural carbohydrates or flavoring agents as additives commonly used in the preparation of beverages. The natural carbohydrates may include conventional sugars, such as monosaccharides (e.g. glucose, fructose, etc.), disaccharides (e.g. maltose, sucrose, etc.) and polysaccharides (e.g., dextrin, cyclodextrin, etc.), and sugar alcohols such as xylitol, sorbitol and erythritol. The natural carbohydrate may be present in a range of 1 to 20% by weight, preferably 5 to 10% by weight, with respect to the total weight of the health food. The flavoring agent may include natural flavoring agents (thaumatin, stevia extract, rebaudioside A, glycyrrhizin, etc.) and synthetic flavoring agents (saccharin, aspartame, etc.). The health food may contain other nutrients, vitamins, minerals (electrolytes), flavors (synthetic or natural flavors), colorants, pectic acids and salts thereof, alginic acids and salts thereof, organic acids, protective colloidal thickeners, pH-adjusting agents, stabilizers, preservatives, glycerin, alcohol, carbonic acid used in carbonated beverages, and the like. In addition, it may contain flesh for the production of natural fruit juices, fruit juice beverages and vegetable beverages. The content of these additives is not particularly limited, but may fall within a range of 0.1 to 20% by weight with respect to the total weight of the health food.

In one aspect of the present invention, the composition for promoting hair growth or preventing hair loss is a cosmetic composition.

In one aspect of the present invention, the composition may be a cosmetic composition. The cosmetic composition of the present invention may be prepared in any one of formulations conventionally prepared in the art, for example, solutions, suspensions, emulsions, pastes, gels, creams, lotions, powders, soaps, shampoo, rinse, hair preparations, surfactant-containing cleansings, oils, powder foundations, emulsion foundations, wax foundations and spray, but is not limited thereto.

In one aspect of the present invention, the composition for promoting hair growth or preventing hair loss is a pharmaceutical composition.

In one aspect of the present invention, the pharmaceutical composition is formulated in the form of any one of injections, powders, granules, tablets, capsules, suspensions, emulsions, syrups, aerosols and external preparations.

The pharmaceutical composition of the present invention may be prepared in the form of a pharmaceutical preparation and a health functional food suitable for oral or parenteral administration and application by further including a suitable vehicle, excipient and/or diluent commonly used in the preparation of pharmaceuticals. In addition, pharmaceutical formulations may be prepared according to conventional methods using the pharmaceutical composition of the present invention. In the preparation of the formulations, the active ingredient may be mixed with the vehicle, diluted with the vehicle, or enclosed in the vehicle in the form of a capsule, sachet or other container. Thus, the formulations may be tablets, pills, powders, capsules, sachets, elixirs, suspensions, emulsions, liquids, syrups, aerosols, soft or hard gelatin capsules, solutions or suspensions for injection, ointments, creams, gels, lotions or the like.

Examples of suitable vehicles, excipients and diluents that can be included in the pharmaceutical compositions of the present invention include lactose, dextrose, sucrose, sorbitol, mannitol, calcium silicate, cellulose, methyl cellulose, microcrystalline cellulose, polyvinylpyrrolidone, water, methylhydroxybenzoate, propylhydroxybenzoate, talc, magnesium stearate and mineral oil. In addition, fillers, anticoagulants, lubricants, wetting agents, fragrances, emulsifiers, preservatives, and the like, which are commonly used in the preparation of formulations, may be further included. The pharmaceutical composition of the present invention may be formulated using methods well known in the art to provide rapid, sustained or delayed release of the active ingredient after administration to a mammal.

Examples of the route of administration of the pharmaceutical composition according to the present invention include, but are not limited to, oral, intravenous, intramuscular, intraarterial, intramedullary, intrathecal, intracardiac, transdermal, subcutaneous, intraperitoneal, intestinal, sublingual or topical administration, or skin application.

The administration and application doses of the pharmaceutical composition of the present invention may vary depending on the patient's condition and body weight, the drug form, the administration route, and the duration of administration, and may be appropriately selected by those skilled in the art. The active ingredient relative to the patient's body weight may range from 0.001 mg/kg to 500 mg/kg, preferably 0.001 to 200 mg/kg. The drug may be administered or applied once a day, or several times in a portionwise manner. The treatment amount does not limit the scope of the present invention in any aspect.

In one aspect of the present invention, the hair loss is stress-induced hair loss.

In one aspect of the present invention, the hair loss is male pattern hair loss.

In one aspect of the present invention, the hair loss is female pattern hair loss.

Hereinafter, the present invention will be described in more detail with reference to specific examples. However, the following examples are provided only for illustration of the present invention, and should not be construed as limiting the scope of the present invention.

### EXAMPLE

### Example 1

### Preparation of Extracts or Fractions of Aster ageratoides Leaves

1.7 L of methanol was added to collected leaves of Aster ageratoides (dry weight of 91 g), and extraction was conducted at room temperature for one week. After this process was repeated three times, the resulting product was filtered and concentrated to dryness with a rotary evaporator at 40° C. to obtain 13.7 g of a methanol extract. The methanol extract was suspended with 140 mL of water and then was extracted with dichloromethane (CH₂Cl₂, 140 ml×3). The aqueous layer was extracted with ethyl acetate (EtOAc, 140 mL×3) to obtain an ethyl acetate fraction. Then, the aqueous layer was again extracted with butanol (BuOH, 140 mL×3) to obtain a butanol fraction.

### Example 2

### Cell Culture and Cell Proliferation Experiment

2-1. Experiment Method

Human dermal papilla cells (hDPCs) used in this experiment were obtained from Cell Bio (Seoul, Korea). The cells were cultured on a tissue culture dish using Dulbecco's modified eagle's medium (DMEM) containing 10% fetal bovine serum and 1% penicillin/streptomycin in an incubator (37° C., 5% CO₂). The medium was changed every 2 to 3 days. The cell proliferation experiment was conducted when the cells reached a confluence of 70%. First, the hDPCs cells were seeded at a density of 1×10⁴ cells/well on a 96-well plate and then incubated in an incubator (37° C., 5% CO₂) for 24 hours. Then, the cells were treated with the sample (5 μg/ml, 50 μg/ml) prepared in Example 1 and minoxidil (10 μM, 100 μM), and were then cultured in an incubator (37° C., 5% CO₂) for 72 hours. After adding 20 μl of 5 mg/ml MTT (3-(4,5-dimethylthiazol-2-yl)-2,5-diphenyl tetrazolium bromide) thereto, the absorbance (540 nm) was measured using an Elisareader (microplate reader-VersaMax System) (Molecular Devices, Sunnyvale, Calif., USA) and compared as a percentage (%) of a control.

2-2. Confirmation of Skin Cell Proliferation Effect

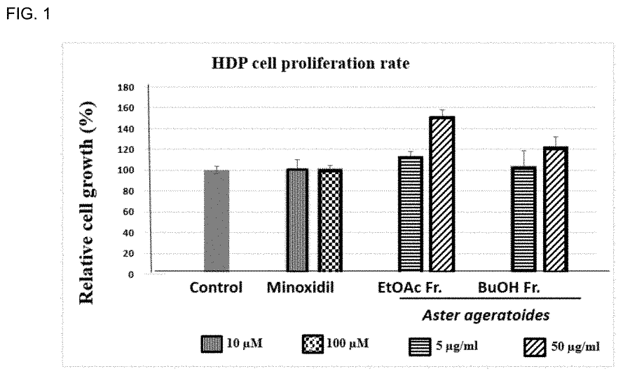

As can be seen from FIG. 1, the groups treated with 5 μg/ml and 50 μg/ml of an ethyl acetate fraction obtained through solvent fractionation from an alcoholic extract of Aster ageratoides exhibited cell proliferation effects of 14% and 52%, respectively, and the group treated with 50 μg/ml of the butanol fraction exhibited a cell proliferation effect of 23%, compared to the negative control group. On the other hand, the groups treated with minoxidil (10 μM, 100 μM) as a positive control exhibited no significant effects.

### Example 3

### Effect of Inducing Hair Growth Phase of Aster ageratoides Extract

3-1. Experiment Method

6-week-old C57BL/6 female mice with white or pink back skin were used as experimental animals and the experiment was conducted according to guidelines for the management and use of laboratory animals approved by the Animal Research Ethics Committee of the Korea Institute of Science and Technology (approval number: KISTIACUC-2018-081). The experimental groups were classified into a total of 4 groups including a negative control group (including a vehicle having no active ingredient) and a positive control group, and 6 mice were used for each group. The back hairs of the mice were depilated using an electric shaver and a test substance was applied to the depilated area once a day for 30 days. The negative control group was treated only with a vehicle (polyethylene glycol: distilled water: ethyl alcohol=5:3:2) used as a solvent for dissolving the reagent, the positive control group was treated with a 3% (w/w) solution of minoxidil (Sigma) in the prepared vehicle, and the sample treatment group was treated with a 1% (w/w) Aster ageratoides extract in the vehicle.

3-2. Confirmation of Effect of Promoting Hair Growth in Skin Tissue

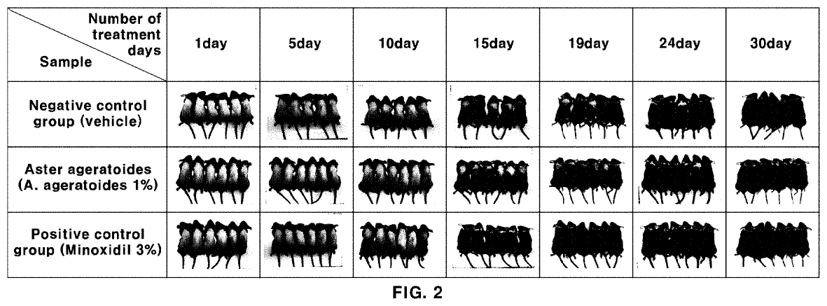

Each sample was applied to the skin once a day at a constant time, and after 30 days, the skin was imaged, the effect of promoting hair growth was compared, and the results are shown in FIG. 2.

As can be seen from FIG. 2, the treatment with the Aster ageratoides extract caused an effect of promoting hair growth. This effect was almost similar to that of the minoxidil (3%) treatment group used as the positive control group. Despite the fact that the Aster ageratoides extract was treated as a mixture of various components at a significantly lower concentration (1%) than the positive control group, the group treated with the Aster ageratoides extract exhibited an effect similar to that of the positive control group. This means that Aster ageratoides exhibited a more potent hair growth promotion effect than minoxidil.

3-3. Effects of Increasing Hair Thickness and Length

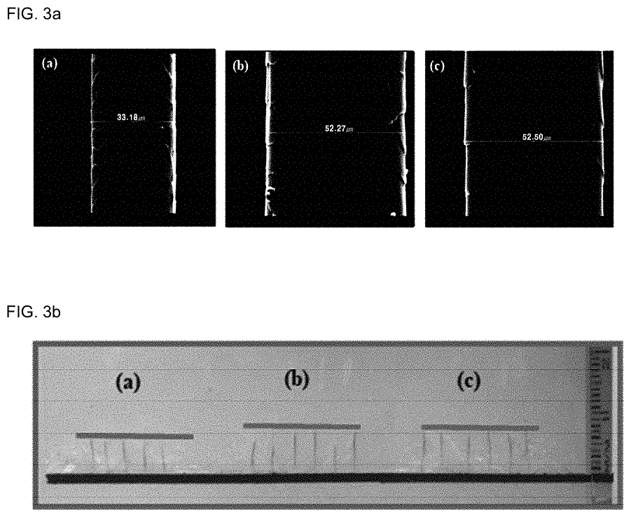

30 days after treatment with the sample, the hairs were randomly collected, the thickness and length of the hairs were measured, and the results are shown in FIG. 3 and Table 1.

As can be seen from FIG. 3A, the group (a) not treated with a drug had a hair thickness of 33.18±2.60 μm, but the group (b) treated with a 1% Aster ageratoides extract had a hair thickness of 49.52±5.03 μm and thus thickened and enriched hairs. This effect was found to be similar to the effect of 53.19±4.04 μm which is a hair thickness obtained by application of 3% minoxidil as the positive control. This demonstrates that the effect of the natural extract at the low concentration was better.

In addition, as can be seen from the comparison in length increase of FIG. 3B, the group (a) not treated with the drug had a hair length of 5 to 6 mm, but the group (b) subjected to application of the 1% Aster ageratoides extract had evenly grown hairs with a length of 7 to 8 mm, which was similar to 6 to 9 mm of the length of hairs treated with the positive control (c).

In conclusion, it can be seen that the effect of promoting hair growth was observed in the group treated with the 1% Aster ageratoides extract and it was indirectly proved that this effect was more potent than that of minoxidil as the positive control.

TABLE 1

| Sample name | Hair width (mm) | Hair length (mm) |
|---|---|---|
| Control (vehicle only) | 33.18 ± 2.60 | 5-6 |
| A. ageratoides 1% | 49.52 ± 5.03 | 7-8 |
| Minoxidil 3% | 53.19 ± 4.04 | 6-9 |

3-4. Confirmation of Hair Growth Effect

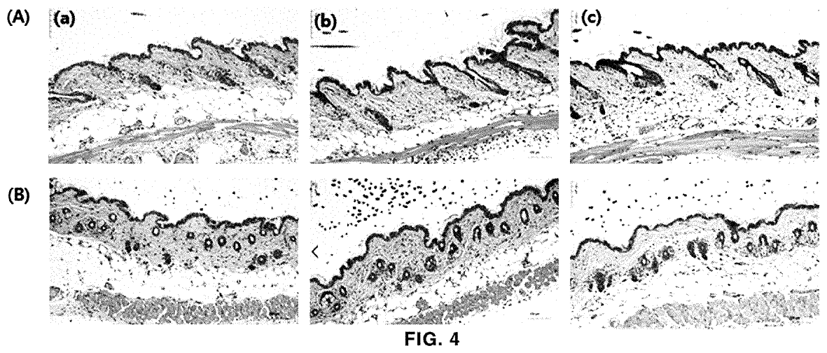

On the last day of application of the test substance, the mice were sacrificed to obtain skin tissues, and then tissues were fixed with paraffin and then prepared into tissue slices. These were stained with hematoxylin and eosin (H&E) and observed under a microscope (100×), and the results are shown in FIG. 4.

As can be seen from FIG. 4, compared to the hair roots of the tissues not treated with a drug (a), the hair roots in the tissues treated with the Aster ageratoides extract (c) grew remarkably, similar to the hair roots of the group treated with minoxidil (b). These results were consistent with the effect of promoting hair growth observed outside the skin of FIG. 2, indicating that the promotion of hair growth was caused by the treatment with the Aster ageratoides extract.

### Example 4

### Inhibitory Effect of Aster ageratoides Extract on Catagen Phase Development

4-1. Experiment Method

6-week-old C57BL/6 female mice with the white or pink back skin were used as experimental animals. The experimental groups were classified into a total of 4 groups including a negative control group (including a vehicle having no active ingredient) and a positive control group, and 6 mice were used for each group. The back hairs of the mice were depilated using an electric shaver and, after 7 days, a test substance was applied to the depilated area once a day for 7 days. 9 days after depilation, 0.1 ml of 0.1% dexamethasone (catagen-inducer agent, Sigma) using propylene glycol as a solvent was applied once to the depilated area once a day for 5 days. After 16 days, the area where growth-phase hairs were maintained among the depilated area was observed. The control group was treated only with a vehicle (polyethylene glycol: distilled water: ethyl alcohol=5:3:2) used as a solvent for dissolving the reagent, the negative control group was treated with 0.1% dexamethasone in the prepared vehicle, the positive control group was treated with a 2% (w/w) solution of minoxidil (Sigma) in the prepared vehicle, and the sample treatment group was treated with a 2% (w/w) Aster ageratoides extract in the vehicle.

4-2. Confirmation of Effect of Prevention of Hair Loss in Catagen Phase

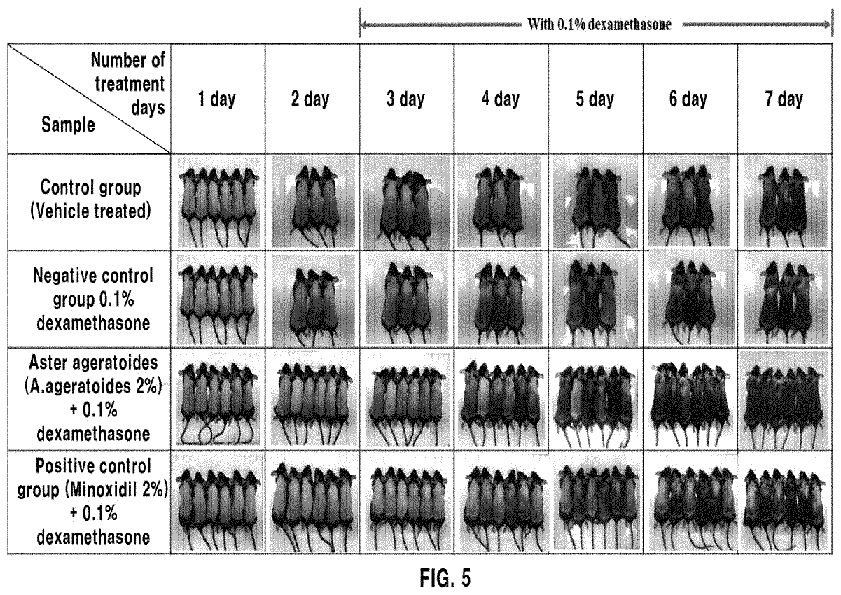

As can be seen from FIG. 5, the effect of preventing catagen-phase hair loss was observed upon treatment with the Aster ageratoides extract. This effect was much better than that of the minoxidil treatment group, which is the positive control group, and hair growth was significantly improved despite dexamethasone treatment.

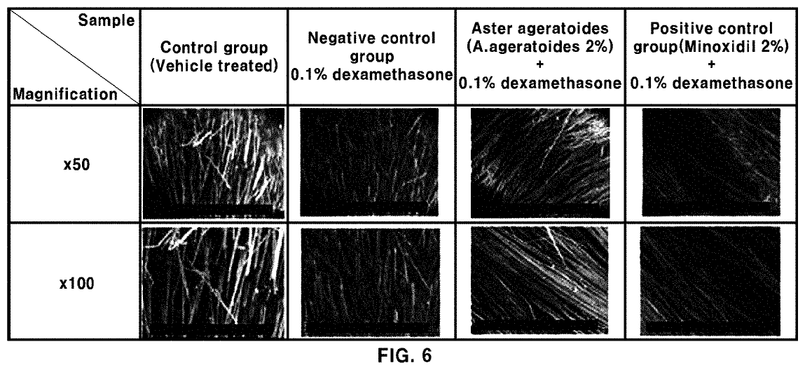

FIG. 6 is an environmental scanning electron microscope (ESEM) image showing changes in the density and thickness of the induced catagen-phase hair by treatment with 0.1% dexamethasone upon application of the 2% Aster ageratoides extract after depilation of 7-week-old C57BL/6 mice. In order to compare the effect, changes in the density and thickness of the hair by the solvent treatment group and 2% minoxidil application were imaged using ESEM and compared. The results showed that, in spite of dexamethasone treatment, the group treated with the Aster ageratoides extract considerably promoted hair growth compared to the group treated with minoxidil, and thus exhibited a much more effective than the same.

### Example 5

### Detection of Antioxidant Effect by Aster ageratoides Extract

5-1. Detection of DPPH Radical Scavenging Effect

Free-radical scavenging activity (Blois et al., Nature, 1958, 181, 1199) was evaluated as IC₅₀ by adding 10 μl of Extracts obtained in Example 1 to 190 μl of a 100 μM 1,1-diphenyl-2-picryl hydrazyl (DPPH) ethanol solution, conducting reaction at 37° C. for 30 minutes and measuring the absorbance at 515 nm. IC₅₀ means a concentration (SC₅₀) at which 50% of free-radical scavenging is obtained when calculating the free-radical scavenging activity. Data are expressed as an average of measurements conducted in triplicate.

5-2. Effect of Inhibiting Formation of Lipid Peroxide by Aster ageratoides Extract

The lipid peroxide production inhibitory effect of the Aster ageratoides extract was tested. Lipid peroxide is a substance produced by peroxidation of lipids through various oxidation reactions. Reactive oxygen species, free radicals and the like oxidize phospholipids of cell membranes containing great amounts of unsaturated fatty acids, producing lipid peroxides in the cell membranes. When the lipid peroxides are accumulated in the cell membranes, the fluidity and functionality of the cell membrane are deteriorated, resulting in local disorders in tissues, such as inhibition of cell functions and changes in cell structures.

Therefore, the effects of inhibiting lipid peroxide production by the Aster ageratoides leaf extract was measured as follows. The animals used in the experiment were male Sprague-Dawley rats, and only water was supplied for 24 hours before the experiment. The experimental animals were subjected to respiratory anesthesia with isoflurane and dissected, and a 0.15 M ice-cold KCl solution was perfused through the liver portal vein to remove blood from the liver and extract the liver. A liver homogenate was prepared by homogenizing with a KCl solution in an amount 10 times the weight of the liver, and the protein concentration was quantified by the Bradford protein method using bovine serum albumin as a standard (Bradford, M M Anal. Biochem. 72, 248, 1976). The lipid peroxidation test was performed using a slightly modified mode of the method of Sanz et al. (Sanz, M. J., et al., Xenobiotica 24, 689-69, 1994). 50 mM Tris-HCl buffer (pH 7.5) was added to 300 μl of the liver homogenate (11 mg protein/ml), 10 μM FeSO₄, 10 μl of a test drug and 0.4 mM ascorbic acid to adjust the total volume to 1 ml, and then the resulting mixture was incubated at 37° C. for 30 minutes. After incubation, 2 ml of a TBA-TCA solution (0.375% thiobarbituric acid, 15% trichloroacetic acid, 0.25 N HCl, 0.01% butylated hydroxytoluene) was added thereto, the mixture was allowed to react at 95° C. for 30 minutes and then cooled and centrifuged (5,000×g) for 10 minutes, and the absorbance of the supernatant was measured at 535 nm. Silymarin, resveratrol and quercetin were used as positive controls to compare the lipid peroxidation inhibitory effect. As a control, DMSO was used, instead of the test drug, and the concentration (IC₅₀) of the sample required to inhibit the formation of lipid peroxide by 50% was measured.

5-3. Detection Results

The results of detecting the antioxidant effects at various concentrations with regard to the alcoholic extract of Aster ageratoides leaves and the dichloromethane fraction, ethylacetate fraction and butanol fraction obtained through solvent fractionation therefrom are shown in Table 2 below.

TABLE 2

| | | Effect | |
|---|---|---|---|
| | | IC₅₀ (μg/m [symbol] ) | |
| | | | Lipid peroxide |
| | | DPPH radical- | production |
| | | scavenging | inhibitory |
| Sample | | activity | activity |
| Methanol extract | | 26.74 ± 2.08 | 64.46 ± 19.52 |
| Dichloromethane fraction | | >50 | 679.80 ± 8.04 |
| Ethyl acetate fraction | | 16.07 ± 2.16 | 51.12 ± 1.69 |
| Butanol fraction | | 19.52 ± 2.34 | 53.15 ± 12.08 |
| Positive | Resveratrol | 56.24 ± 5.22 | 35.64 ± 0.01 |
| controls | Quercetin | 19.04 ± 2.25 | 29.57 ± 1.32 |
| (μM) | Ascorbic acid | 29.27 ± 2.49 | — |
| | Trolox | 47.50 ± 0.41 | >100 |
| | Silymarin (μg/m [symbol] ) | 43.22 ± 2.58 | 98.7 ± 0.02 |

*Data are mean ± standard error

As can be seen from Table 2, the Aster ageratoides extract or the solvent fractions thereof exhibit a DPPH-radical scavenging effect and a lipid peroxide production inhibitory effect, which were found to be antioxidant effects effective for stress prevention. That is, the ethyl acetate fraction and the butanol fraction, which are solvent fractions from the Aster ageratoides alcohol extract, have 50% DPPH radical-scavenging activity (SC₅₀) of 16.07 to 19.52 μg/ml, and thus exhibit an excellent effect compared to the DPPH radical-scavenging activity (SC₅₀) of ascorbic acid, quercetin and resveratrol, which are well-known antioxidant substances, as positive controls. Regarding the effect of inhibiting the production of lipid peroxide using rat liver homogenates, the ethyl acetate fraction solvent-fractionated from the Aster ageratoides alcohol extract exhibited a better inhibitory effect than that of silymarin used as a liver-protective agent.

As described above, the Aster ageratoides extract or each of solvent fractions thereof according to the present invention exhibits excellent effects of proliferating human hair follicle dermal papilla cells, of increasing the thickness and length of hair, and of enhancing hair growth as observed upon visual inspection of the skin, of strengthening hair roots as observed upon skin tissue inspection, and of preventing hair loss in an artificially induced regression phase. In addition, the Aster ageratoides extract or each of solvent fractions thereof according to the present invention was proved to be excellent in preventing hair loss due to stress due to the excellent antioxidant effect thereof. Therefore, the Aster ageratoides extract or each of solvent fractions thereof according to the present invention is useful as an active ingredient for cosmetic compositions for promoting hair growth and for preventing, ameliorating and treating hair loss, or for pharmaceutical compositions or health food compositions for preventing, ameliorating and treating scalp diseases or disorders.

[Preparation Example]

Meanwhile, the pharmaceutical composition containing the Aster ageratoides extract or solvent fraction thereof according to the present invention can be prepared in various forms according to the purpose thereof. The following Preparations 1 to 4 illustrate a method for preparing a drug containing, as an active ingredient, the Aster ageratoides extract or solvent fraction thereof according to the present invention, but the present invention is not limited thereto.

Preparation 1: Tablets (Direct Compression)

5.0 mg of an active ingredient was sieved and mixed with 14.1 mg of lactose, 0.8 mg of crospovidone USNF and 0.1 mg of magnesium stearate, and the mixture was compressed into tablets.

Preparation 2: Tablets (Wet Granulation)

5.0 mg of an active ingredient was sieved and was mixed with 16.0 mg of lactose and 4.0 mg of starch. 0.3 mg of Polysorbate 80 was dissolved in pure water, and an appropriate amount of the resulting solution was added to the mixture, followed by granulation. The granules were dried, sieved, and mixed with 2.7 mg of colloidal silicon dioxide and 2.0 mg of magnesium stearate. The granules were compressed into tablets.

Preparation 3. Powders and Capsules

5.0 mg of an active ingredient was sieved and then mixed with 14.8 mg of lactose, 10.0 mg of polyvinylpyrrolidone and 0.2 mg of magnesium stearate. Hard No. 5 gelatin capsules were filled with the resulting mixture using an appropriate device.

Preparation 4. Injections

Injections were prepared by incorporating 100 mg of the active ingredient as well as 180 mg of mannitol, 26 mg of Na₂HPO₄.12H₂O and 2,974 mg of distilled water.

In addition, the health food composition containing the Aster ageratoides extract or solvent fraction thereof according to the present invention can be prepared in various forms according to the purpose thereof. The following Preparations 5 to 9 illustrate a method for preparing a health food containing, as an active ingredient, the Aster ageratoides extract or solvent fraction thereof according to the present invention, but the present invention is not limited thereto.

Preparation 5. Granular Health Foods

1,000 mg of an active ingredient, 70 μg of vitamin A acetate, 1.0 mg of vitamin E, 0.15 mg of vitamin B₂, 0.5 mg of vitamin B₆, 0.2 μg of vitamin B₁₂, 10 mg of vitamin C, 10 μg of biotin, 1.7 mg of nicotinamide, 50 μg of folic acid, 0.5 mg of calcium pantothenate, 1.75 mg of ferrous sulfate, 0.82 mg of zinc oxide, 25.3 mg of magnesium carbonate, 15 mg of potassium phosphate monobasic, 55 mg of dibasic calcium phosphate, 90 mg of potassium citrate, 100 mg of calcium carbonate, and 24.8 mg of chloride magnesium were mixed and then a granular health food was prepared according to a conventional method.

Preparation 6. Health Drink

1,000 mg of an active ingredient, 1,000 mg of citric acid, 100 g of oligosaccharide, 2 g of a plum concentrate and 1 g of taurine were mixed, and purified water was added thereto to adjust the total volume to 900 ml.

After stirring and heating at 85° C. for about 1 hour, the resulting solution was filtered and charged in a sterilized 2 liter container, and the container was sealed and sterilized to prepare a health drink.

Although the composition ratio above is obtained as a mixture of components suitable for preferred drinks in a preferred example, the mixing ratio may be arbitrarily modified according to regional and ethnic preferences, such as target customers, target county and usage.

Preparation 7. Flour-Based Foods

0.5 to 5 g of an active ingredient was added to 100 g of flour, and bread, cakes, cookies, crackers and noodles were prepared using the resulting mixture to obtain foods for health improvement.

Preparation 8. Dairy Products

5 to 10 g of an active ingredient was added to 100 g of milk, and various dairy products such as butter and ice cream were prepared using the milk.

Preparation 9. Sunsik (Korean Ready-to-Eat Food Prepared from Grains)

30 g of brown rice, 20 g of barley, 10 g of glutinous rice, and 15 g of adlay were pregelatinized by a known method, dried, and roasted and then prepared into a powder having a particle size of 60 mesh with a grinder. 7 g of black beans, 7 g of black sesame seeds and 7 g of perilla seeds were steamed by a known method, and then dried, roasted and then prepared into a powder having a particle size of 60 mesh with a grinder. The grains and the seeds prepared as described above were mixed with 3 g of the active ingredient of the present invention to prepare Sunsik.

As is apparent from the foregoing, the composition according to an embodiment of the present invention has an effect of preventing hair loss.

The composition according to an embodiment of the present invention has an effect of promoting hair growth.

The composition according to an embodiment of the present invention has an effect of proliferating human hair follicle dermal papilla cells (hDPCs).

In an embodiment, the present invention has an effect of providing a food composition for preventing hair loss and promoting hair growth.

In an embodiment, the present invention has an effect of providing a pharmaceutical composition for preventing hair loss and promoting hair growth.

In an embodiment, the present invention has an effect of providing a cosmetic composition for preventing hair loss and promoting hair growth.

The methanol extract of Aster ageratoides leaves and the solvent fraction obtained by solvent fractionation therefrom obtained through the present invention have excellent effects of promoting hair growth and preventing hair loss. Therefore, the present invention provides a food composition, cosmetic composition or pharmaceutical composition for protecting the scalp and hair containing, as an active ingredient, the methanol extract of Aster ageratoides leaves and the solvent fraction thereof.

Accordingly, the methanol extract of Aster ageratoides leaves and the solvent fraction obtained by solvent fractionation therefrom obtained through the present invention promotes proliferation of human hair follicle dermal papilla cells (hDPCs) and prevents hair loss in a regression phase, thus being used for a cosmetic composition or pharmaceutical composition for protecting the scalp and hair.

The effects of the present invention are not limited to those mentioned above. It should be understood that the effects of the present invention include all effects that can be inferred from the description of the present invention.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A composition for promoting hair growth or preventing hair loss containing an Aster ageratoides extract or a fraction thereof as an active ingredient.

2. The composition according to claim 1, wherein the Aster ageratoides extract is an extract of an aboveground or underground part of Aster ageratoides.

3. The composition according to claim 1, wherein the extract is an extract obtained through extraction using water, C₁-C₅ alcohol, dichloromethane, acetone, an aqueous acetone solution or an aqueous C₁-C₅ alcohol solution.

4. The composition according to claim 3, wherein concentrations of the aqueous C₁-C₅ alcohol solution and the aqueous acetone solution are each independently 10% to 90% (v/v).

5. The composition according to claim 1, wherein the fraction is an ethyl acetate fraction of an Aster ageratoides C₁-C₅ alcohol extract.

6. The composition according to claim 1, wherein the fraction is a butanol fraction of the Aster ageratoides C₁-C₅ alcohol extract.

7. The composition according to claim 2, wherein the extract is an extract of leaves of Aster ageratoides.

8. The composition according to claim 1, wherein the composition promotes hair growth or prevents hair loss by promoting proliferation of human hair follicle dermal papilla cells.

9. The composition according to claim 1, wherein the composition promotes hair growth or prevents hair loss by inhibiting a regression phase (catagen) of hairs.

10. The composition according to claim 1, wherein the composition promotes hair growth or prevents hair loss by growing hair roots.

11. The composition according to claim 1, wherein the composition promotes hair growth or prevents hair loss by increasing a hair thickness or a hair length.

12. The composition according to claim 1, wherein the composition promotes hair growth or prevents hair loss through free-radical scavenging or lipid peroxidation inhibition.

13. A method for promoting hair growth or preventing hair loss of a subject, wherein the method comprises administering an effective amount of an Aster ageratoides extract or a fraction thereof to the subject in need thereof.

14. The method according to claim 13, wherein the Aster ageratoides extract is an extract of an aerial or underground part of Aster ageratoides.

15. The method according to claim 13, wherein the extract is an extract obtained through extraction using water, C₁-C₅ alcohol, dichloromethane, acetone, an aqueous acetone solution or an aqueous C₁-C₅ alcohol solution, and
wherein concentrations of the aqueous C₁-C₅ alcohol solution and the aqueous acetone solution are each independently 10% to 90% (v/v).

16. The method according to claim 13, wherein the fraction is a butanol fraction of the Aster ageratoides C₁-C₅ alcohol extract.

17. The method according to claim 14, wherein the extract is an extract of leaves of Aster ageratoides.

18. The method according to claim 13, wherein the Aster ageratoides extract or a fraction thereof is administered in a form of a health functional food composition.

19. The method according to claim 13, wherein the Aster ageratoides extract or a fraction thereof is administered in a form of a cosmetic composition.

20. The method according to claim 13, wherein the Aster ageratoides extract or a fraction thereof is administered in a form of a pharmaceutic composition.
